(19) United States
(12) Patent Application Publication
(10) Pub. No.: US 2020/0297288 A1
(43) Pub. Date: Sep. 24, 2020

(54) SYSTEM FOR VISUALIZING BIOSIGNAL AND METHOD OF EXTRACTING EFFECTIVE PATTERN

(71) Applicant: KOREA INSTITUTE OF SCIENCE & TECHNOLOGY INFORMATION, Daejeon (KR)

(72) Inventors: Seonho KIM, Seoul (KR); Hong-Woo CHUN, Seoul (KR); Jungjoon KIM, Seoul (KR); Byoung-Youl COH, Seoul (KR)

(21) Appl. No.: 16/825,280

(22) Filed: Mar. 20, 2020

(30) Foreign Application Priority Data
Mar. 21, 2019 (KR) 10-2019-0032381

(51) Int. Cl. A61B 5/00; G06K 9/00

(52) U.S. Cl. CPC A61B 5/7275; A61B 5/742; A61B 5/04004; G06K 9/00523; G06K 9/0055; A61B 5/7264

(57) ABSTRACT

Provided are a biosignal visualizing system, which may easily learn a biosignal, may easily make a diagnosis, and may perform analysis in real time, and an effective pattern extracting method using the same, in order to determine a disease using a biosignal via deep learning.

### CROSS-REFERENCE TO RELATED APPLICATION

This application claims priority under 35 U.S.C. § 119(a) to Republic of Korea Patent Application No. 10-2019-0032381 filed on Mar. 21, 2019, which is incorporated by reference herein in its entirety.

### BACKGROUND OF THE INVENTION

### 1. Field of the invention

The present disclosure relates to a biosignal visualizing system and an effective pattern extracting method, and particularly, to a technology that visualizes a biosignal so as to extract a pattern effective for making a diagnosis of a disease.

### 2. Description of the Prior Art

Research on a technology that measures various physical conditions of human bodies by using biosignals is being conducted.

A biosignal may include, for example, an electroencephalogram (EEG) (or brainwaves), an electromyogram (EMG), an electrocardiography (ECG), or the like.

An EEG refers to a waveform obtained by measuring a subtle change in electric potential using an electrode attached to the scalp, under the condition that a stimulus is applied to the cerebral cortex, an ionized current flows among nerve cells, and an electric field and a magnetic field are formed. Particularly, the EEG is distributed in a frequency band of 0 to 100+ Hz, and an electric potential change is about dozens of μV. Accordingly, the EEG may be obtained by amplifying the electric potential change.

Diseases may be determined by learning the biosignal measured in this manner. A technology that learns biosignals to determine diseases may include a technology that analyzes diseases using biosignals themselves, and a machine learning technology using artificial intelligence such as deep learning or the like.

Among them, the machine learning technology using deep learning learns biosignals of a patient group and a non-patient group, and determines whether a person has a disease via learning.

However, in the learning process of the machine learning technology that uses deep learning, how biosignals are learned, how a diagnosis is made using a patient biosignal and a non-patient biosignal, and the like are blackboxed. Therefore, it is inappropriate for a medical diagnosis that requires a sufficient description when making a decision, which is a drawback.

Also, the machine learning technology may be difficult to perform real-time analysis since it is very complex to calculate the biosignal learning and diagnosis. Also, the machine learning technology does not have a function of modeling and visualizing biosignal data, which may allow easy recognition of the difference between a non-patient and a patient.

### SUMMARY OF THE INVENTION

The present disclosure has been made in order to solve the above-mentioned problems in the prior art and an aspect of the present disclosure is to provide a biosignal visualizing system and an effective pattern extracting method, which may learn biosignals of a patient and a non-patient, visualize the biosignals, obtain the difference between two groups via comparison, and visually identify whether a predetermined person has a disease, using a biosignal of the predetermined person.

In accordance with an aspect of the present disclosure, a biosignal visualizing system may include: a pattern expression unit configured to express a learning biosignal as multiple patters according to a predetermined condition; an identification unit configured to determine whether the multiple patterns are effective patterns, and to identify pattern information of the effective patterns; a measurement unit configured to measure the value of a probability that different patterns neighboring in the multiple patterns are adjacent to each other; and a display unit configured to display the multiple patterns on a matrix including columns and rows, according to the probability values.

The pattern expression unit is configured to obtain non-patient patterns and patient patterns, which are obtained by expressing a non-patient biosignal and a patient biosignal of the learning biosignal as multiple patterns according to a predetermined condition, the identification unit is configured to determine whether the non-patient patterns and the patient patterns are effective patterns, and to identify effective non-patient pattern information and effective patient pattern information respectively from the non-patient patterns and the patient patterns, the measurement unit is configured to obtain a non-patient pattern probability value indicating a probability that different patterns neighboring in the multiple non-patient patterns are adjacent to each other, and a patient pattern probability value indicating a probability that different patterns neighboring in the multiple patient patterns are adjacent to each other, and the display unit is configured to display the non-patient pattern probability value and the patient pattern probability value on matrices.

The system may further include an extraction unit configured to extract a mismatch pattern where the non-patient pattern probability value and the patient pattern probability value do not match, so as to extract a disease pattern associated with a disease that a patient has.

The extraction unit is configured to extract the mismatch pattern using an exclusive-or (XOR) operation.

The pattern expression unit is configured to obtain predetermined person patterns obtained by expressing a predetermined person biosignal as multiple patterns according to a predetermined condition, the identification unit is configured to identify whether the predetermined person patterns are effective patterns, and to identify effective predetermined person pattern information from the predetermined person patterns, the measurement unit is configured to obtain a predetermined person pattern probability value indicating a probability that different patterns neighboring in the multiple predetermined person patterns are adjacent to each other, the display unit is configured to display the predetermined person pattern probability value on a matrix, and the extraction unit is configured to extract whether a pattern that matches the disease pattern is retained in the predetermined person patterns.

The system may further include a prediction unit configured to predict a disease of the predetermined person depending on whether the pattern that matches the disease pattern is retained in the predetermined person patterns.

The system may further include a pattern color determination unit configured to display the multiple patterns in different colors according to a predetermined condition

The system may further include: a unit determination unit configured to segment the learning biosignal and the predetermined person biosignal, based on a predetermined unit size according to a predetermined condition; and a pattern assignment unit configured to assign a pattern to each predetermined unit.

The number of the patterns increases as the unit size used for segmenting the learning biosignal and the predetermined person biosignal increases.

In accordance with an aspect of the present disclosure, an effective pattern extracting method may include: expressing a learning biosignal as multiple patterns according to a predetermined condition; identifying whether the multiple patterns are effective patterns, and identifying pattern information of the effective patterns; measuring a value of a probability that different patterns neighboring in the multiple patterns are adjacent to each other; and displaying the multiple patterns on a matrix including columns and rows, according to the measured probability values.

The operation of expressing the learning biosignal as the multiple patterns according to the predetermined condition includes expressing a non-patient biosignal and a patient biosignal of the learning biosignal as non-patient patterns and patient patterns, the operation of identifying whether the multiple patterns are effective patterns, and the identifying the pattern information of the effective pattern includes determining whether the non-patient patterns and the patient patterns are effective patterns, and identifying effective non-patient pattern information and effective patient pattern information respectively from the non-patient patterns and the patient patterns, the operation of measuring the value of the probability that different patterns neighboring in the multiple patterns are adjacent to each other includes obtaining a patient pattern probability value and a non-patient pattern probability value, the operation of displaying the multiple patterns on a matrix including columns and rows according to the probability values includes displaying the non-patient pattern probability value and the patient person pattern probability value on matrices, and the method includes extracting a mismatch pattern where the non-patient probability value and the patient person pattern probability value do not match, so as to extract a disease pattern associated with a disease that a patient has.

The operation of extracting the disease pattern associated with the disease that the patient has further includes: extracting whether a pattern that matches the disease pattern is retained in predetermined patterns extracted in association with a predetermined person.

The operation of displaying the multiple patterns on a matrix including columns and rows, according to the probability values includes: displaying the multiple patterns in different colors, according to a predetermined condition.

A biosignal visualizing system of the present disclosure patternizes a non-patient biosignal and a patient biosignal, displays the same in color on square orthogonal coordinates, so that non-patient patterns and patient patterns can be visualized and a disease pattern can be extracted from the visualized non-patient patterns and patient patterns. By extracting whether a disease pattern exists, a pattern effective for predicting a disease and making a diagnosis may be visually identified as detailed information, which is advantageous.

In addition, after learning the non-patient patterns and the patient patterns, the system compares the patient patterns and the non-patient patterns with predetermined person patterns, obtained by patterninzing a predetermined person biosignal and displaying the same in color on the square orthogonal coordinates, identifies whether a disease pattern is retained in the predetermined person patterns, and identifies detailed information associated with whether the predetermined person has a disease.

### BRIEF DESCRIPTION OF THE DRAWINGS

The above and other aspects, features, and advantages of the present disclosure will be more apparent from the following detailed description taken in conjunction with the accompanying drawings, in which:

FIG. 1 is a schematic diagram of a biosignal visualizing system according to an embodiment of the present disclosure;

FIG. 2 is a diagram illustrating a process of expressing a biosignal as multiple patterns according to an embodiment of the present disclosure;

FIG. 3 is a diagram illustrating a Gaussian curve obtained by analyzing a biosignal according to an embodiment of the present disclosure;

FIGS. 4A, 4B, and 4C are diagrams illustrating examples of a biosignal which is displayed as being expressed as multiple patterns on a matrix according to an embodiment of the present disclosure;

FIG. 5 is a flowchart illustrating a biosignal visualizing method according to an embodiment; and

FIG. 6 is a flowchart illustrating a biosignal visualizing method according to an embodiment of the present disclosure.

### DETAILED DESCRIPTION OF THE EXEMPLARY EMBODIMENTS

Hereinafter, an embodiment of the present disclosure will be described with reference to the accompanying drawings.

Although a disease described in an embodiment of the present disclosure is considered as dementia, it is apparent that overall diseases associated with the human body are predictable, in addition to the dementia.

Also, a biosignal described in an embodiment of the present disclosure may be one of an electroencephalogram (hereinafter, brainwaves), electrocardiographic waves, and the like, and the present disclosure is not limited by the type of biosignal.

Also, a biosignal visualizing system described in an embodiment of the present disclosure will be described with reference to a system which is implemented via an artificial intelligence device that is capable of performing deep learning.

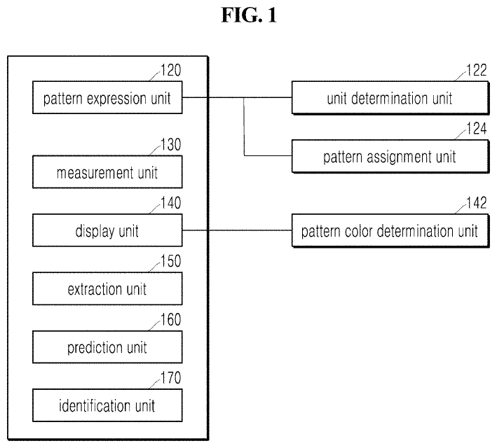

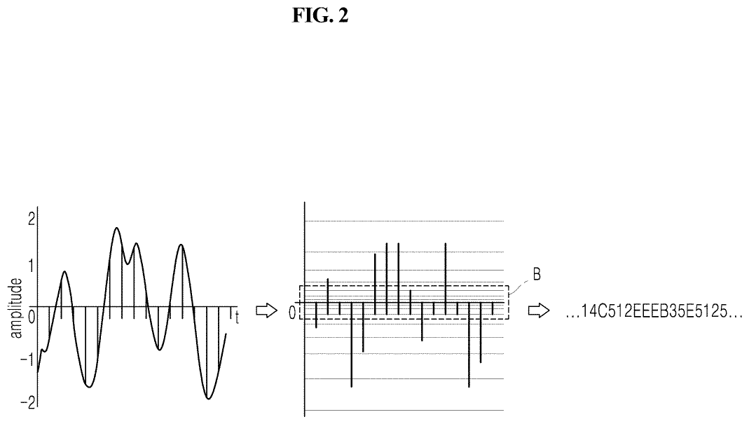

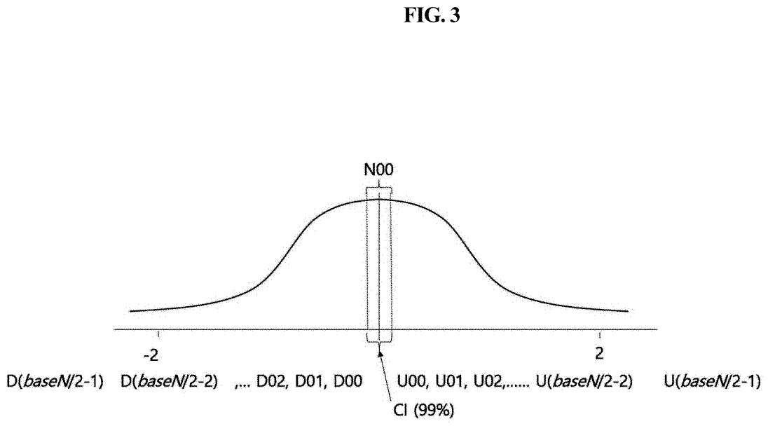

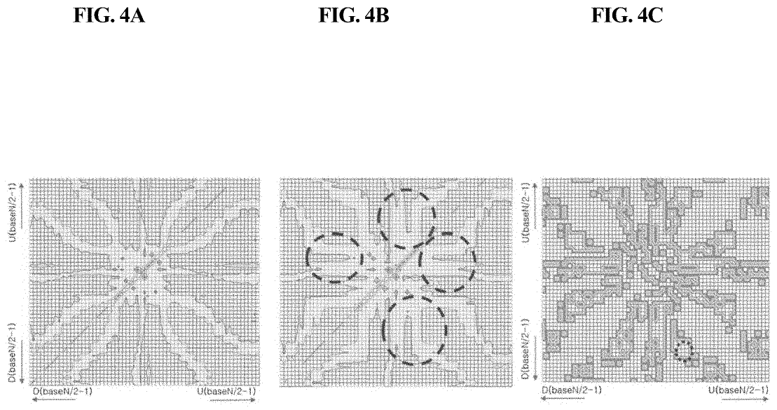

FIG. 1 is a schematic diagram of a biosignal visualizing system according to an embodiment of the present disclosure. FIG. 2 is a diagram illustrating a process of expressing a biosignal as multiple patterns according to an embodiment of the present disclosure. FIG. 3 is a diagram illustrating a Gaussian curve obtained by analyzing a biosignal according to an embodiment of the present disclosure. FIGS. 4A, 4B, and 4C are diagrams illustrating examples of a biosignal which is displayed as being expressed as multiple patterns on a matrix according to an embodiment of the present disclosure.

Before providing a detailed description with reference to drawings, note that a deep learning technology is a technology used for grouping or classifying objects or data. Particularly, machine learning refers to a technology in which machine interprets data and automatically obtain an optimal feature. The machine learning may have a significantly high accuracy in detecting an optimal feature, which is advantageous.

Among the machine learning technologies, the deep learning technology has the best performance. However, the process of learning biosignals and determining whether a predetermined biosignal is a patient biosignal or a non-patient biosignal is blackboxed and is not describable. Accordingly, the deep learning technology is inappropriate for a medical diagnosis device which requires a detailed description.

Particularly, the deep learning technology does not expose how a biosignal is learned and a criterion for determining whether an input biosignal corresponds to a patient or a non-patient. Therefore, the criterion for determining a patient according to a biosignal is unclear, and the deep learning technology is inappropriate for a medical diagnosis machine that requires a description when making a decision.

Hereinafter, an embodiment of the present disclosure provides a biosignal visualizing system that is capable of learning a biosignal such as brainwaves, electrocardiogram, or the like via machine learning which is the deep learning technology, and is capable of visually identifying the probability of disease such as dementia or the like based on the learned information.

Particularly, referring to FIG. 1, a biosignal visualizing system according to an embodiment of the present disclosure may include a pattern expression unit 120, a measurement unit 130, a display unit 140, and an identification unit 170.

The pattern expression unit 120 may be configured to express a teaming biosignal, which may be learned, as multiple patterns according to a predetermined condition.

Particularly, a learning biosignal, which may be teamed, is input to a device in which the biosignal visualizing system is implemented. In this instance, the device that implements the biosignal visualizing system may not learn the input teaming biosignal as it is, but may simplify and learn the same.

To this end, if a learning biosignal is input to the biosignal visualizing system, the system may segment and patternizes the learning biosignal according to a predetermined time and condition

To this end, the biosignal visualizing system may include a unit determination unit 122 configured to segment an input teaming biosignal according to a predetermined unit size according to a predetermined condition.

The unit determination unit 122 may include a condition used for segmenting a teaming biosignal. The condition for segmenting a teaming biosignal included in the unit determination unit 122 may be one of the various conditions depending on the type of input biosignal, the type of disease to be predicted, and the like. According to an embodiment of the present disclosure, an example of segmentation performed according to a predetermined time will be described.

Also, a learning biosignal segmented according to a predetermined time and condition may be expressed using various patterns, and a pattern may be one of the various patterns such as a line, a figure, and the like.

Each learning biosignal segment may be expressed as a pattern. Each pattern may be one of a number, a character, and a combination of a number and a character, as illustrated in FIG. 2. Hereinafter, an embodiment of the present disclosure provides an example of a combination of a number and an English character.

In the process of patterninzing a learning biosignal, a pattern may be differently expressed according to the type of biosignal (e.g., brainwaves, ECG, and the like), a condition used for pattern segmentation, and the like.

To this end, an input learning biosignal may be analyzed, and a condition for patterninzing the learning biosignal may be set according to the analyzed result. For example, a pattern and a variation used for determining a learning biosignal unit may be determined in order to patternize a learning biosignal.

By analyzing a learning biosignal unit according to the determined condition, a Gaussian curve (normal distribution) as illustrated in FIG. 3 may be obtained. In the obtained Gaussian curve, baseN denotes the number of patterns to be applied. It may be configured to maximize the number of patterns distributed in a part close to the center 0 of the Gaussian curve, and to minimize the number of patterns distributed in a part close to the edge of the Gaussian curve.

The biosignal visualizing system may further include a pattern assignment unit 124 that assigns a pattern to each predetermined unit of a segmented learning biosignal. A condition used for assigning a pattern to each predetermined unit of a segmented learning biosignal may be one of the various conditions according to the type of biosignal, a visualizing method, and the like.

As described above, when a learning biosignal is expressed as multiple patterns, the identification unit 170 may determine whether the multiple expressed patterns are effective patterns, and may extract information associated with effective patterns.

Particularly, the identification unit 170 may identify an effective pattern, that is, a pattern that has information used for making a diagnosis or estimating a disease among the multiple patterns.

Also, the identification unit 170 may identify information associated with the identified effective pattern. In order to identify the information associated with the effective pattern, information retained in the identified effective pattern may be identified via a pattern information storage unit generated during the process of patterninzing the learning biosignal (e.g., a dictionary that stores information indicated by different patterns).

For example, it is assumed that the learning biosignal is patternized as “ . . . 14C512EEEB . . . ” and <<EB>> thereof is identified as being an effective pattern. Here, it is assumed that <<EB>> is an effective pattern, which may be identified when a biosignal of a dementia patient is patternized. The information may be stored in the pattern storage unit, and the identification unit 170 compares pattern information stored in the pattern information storage unit and multiple patterns extracted from the learning biosignal, and may identify whether the effective pattern <<EB>> is included in the learning biosignal. If it is identified that the learning biosignal includes the pattern <<EB>>, information associated with the effective pattern <<EB>> may be identified based on the information stored in the pattern information storage unit.

As described above, if an effective pattern is identified from the patternized learning biosignal, the measurement unit 130 may measure the value of the probability that different patterns neighboring in the multiple patterns will be adjacent to each other.

Particularly, referring to FIG. 2, if it is assumed that a patternized learning biosignal is “ . . . 14C512EEEB . . . ”, the measurement unit 130 may measure the value of the probability that pattern E and pattern E will be adjacent to each other, the value of the probability that pattern E and pattern B will be adjacent to each other, and the like.

In this instance, a probability value may be measured in consideration of the order of neighboring patterns, according to a condition. For example, the value of the probability that pattern E and pattern B will be adjacent to each other may be measured only if the pattern E and pattern B are sequentially identified.

Unlike the above, only the value of the probability that different patterns will be adjacent to each other may be measured without taking into consideration the order of patterns. That is, only the value of the probability that pattern E and pattern B will be adjacent to each other may be measured by assuming that the case in which the patterns are aligned in the order of E and B, and the case in which the patterns are aligned in the order of B and E are the same.

The condition used for obtaining a probability value may be different depending on the type of biosignal, the number of patterns, and the like, and the present disclosure is not limited by the condition used for obtaining a probability value.

As described above, if the value of the probability that different patterns will be adjacent to each other is measured, the display unit 140 may display the multiple patterns on a matrix including columns and rows, according to measured probability values.

As described above, the value of the probability that different patterns will be adjacent to each other may be measured on condition that measurement is performed according to the order of different patterns, and the value of the probability that different patterns will be adjacent to each other may be measured on condition that measurement is performed without taking into consideration the order of different patterns.

If the probability values measured according to the conditions are displayed on matrices, the matrices which are different in shape according to the conditions may be obtained.

For example, a matrix obtained on condition that measurement is performed without taking into consideration the order of different patterns may be implemented as a matrix which is symmetrical about a diagonal like a matrix illustrated in FIG. 4.

If an insufficient number of patterns are obtained, a probability value is measured without taking into consideration the order of patterns, so as to increase the number of patterns and to sufficiently visualize a biosignal.

Unlike the same, a matrix obtained on condition that a probability value is measured in the order of different patterns may be implemented as a matrix which is not symmetrical.

Meanwhile, the probability that pattern E and pattern B will be adjacent to each other and the probability that pattern B and pattern E will be adjacent to each other may be different from each other, when a probability value is measured according to the order of patterns. If an effective pattern is <<EB>>, accurate information associated with the effective pattern (effective pattern information) may be obtained, which is advantageous.

Referring again to FIG. 4, the value of the probability that different patterns will be adjacent to each other may be displayed in color on a matrix. That is, if it is assumed that the probability that pattern A and pattern A will be adjacent to each other is 1, the probability that pattern A and pattern B will be adjacent to each to other is less than the probability that pattern A and pattern A will be adjacent to each other. The probability that pattern A and pattern C will be adjacent to each other is less than the probability that pattern A and pattern B will be adjacent to each other.

To display the measured probability values on the matrix partitioned by columns and rows, the matrix is gridded to include coordinates. The coordinates may include coordinates A to Z, may include coordinates 0 to 9, or may include coordinates based on combinations of numbers and English patterns.

The matrix gridded to include coordinates may include square orthogonal coordinates, and the value of the probability of adjacency for each pattern may be displayed in color on the square orthogonal coordinates. For example, it is assumed that pattern A is a start coordinate on the square orthogonal coordinates and the value of the probability that pattern A and pattern A will be adjacent to each other is 1, the value of the probability that pattern A and pattern A will be adjacent to each other may be displayed in red on the square orthogonal coordinates. The value of the probability that pattern A and pattern B will be adjacent to each other may be different from the value of the probability that pattern A and pattern A will be adjacent to each other, and the probability value that pattern A and pattern B will be adjacent to each other may be displayed in blue, which is different from the color corresponding to the value of the probability that pattern A and pattern A will be adjacent to each other, on the square orthogonal coordinates.

The biosignal visualizing system according to an embodiment of the present disclosure may further include a pattern color determination unit 142, so as to display multiple patterns in different colors according to a predetermined condition, when displaying the patterns on a matrix.

It is preferable that colors corresponding to probability values from the probability value that pattern A and pattern A will be adjacent to each other to the probability value that pattern A and pattern Z will be adjacent to each other are determined in advance, and the pattern color determination unit 142 is configured to automatically display color on the matrix according to a probability value extracted from a pattern extracted from an input biosignal. A learning biosignal may be classified as a non-patient biosignal and a patient biosignal. Accordingly, the pattern expression unit 120 may obtain non-patient patterns and patient patterns which are obtained by expressing the non-patient biosignal and the patient biosignal as multiple patterns according to a predetermined condition.

If the non-patient patterns and patient patterns are obtained, the measurement unit 130 may obtain a non-patient pattern probability value indicating the probability of adjacency for each of the obtained multiple non-patient patterns, and may obtain a patient pattern probability value indicating the probability of adjacency for each of the obtained multiple patient patterns.

Subsequently, the display unit 140 displays the non-patient pattern probability values (see FIG. 4A) and the patient pattern probability values (see FIG. 4B) on the square coordinates of matrices, respectively.

By displaying the non-patient pattern probability values and the patient pattern probability values on the square coordinates of the matrices, a part (an area marked by a circle in FIG. 4B, a mismatch pattern) corresponding to a mismatch between the non-patient pattern probability values and the patient pattern probability values may be extracted.

To this end, the biosignal visualizing system may further include an extraction unit 150 configured to extract a mismatch pattern.

Particularly, the extraction unit 150 may extract a mismatch pattern via a first matrix (see FIG. 4A) on which the non-patient patterns are displayed and a second matrix (see FIG. 4B) on which the patient patterns are displayed, may identify the patient pattern probability value of the mismatch pattern, and may extract a pattern (an area marked by a circle of FIG. 4C) according to the identified probability value. That is, the name of the pattern having the probability value of the mismatch pattern may be extracted.

The extraction unit 150 may extract the mismatch pattern by using the exclusive-or (XOR) operation. The XOR operation is an operation that gives a result of 0 when input variables have the same bit, and otherwise, it gives a result of 1. The mismatch pattern may be extracted based on an operation that gives result of 1.

As described above, a mismatch pattern may be extracted from extracted non-patient patterns and patient patterns, and the extracted mismatch pattern is referred to as a disease pattern associated with a disease that a patient has. The disease pattern may be used as a criterion for estimating whether a predetermined person is a patient depending on whether the pattern same as the disease pattern is retained in predetermined person patterns extracted from a biosignal of the predetermined person.

Accordingly, a patient biosignal and a non-patient biosignal are segmented, non-patient biosignal segments and patient biosignal segments are patternized, the value of the probability of adjacency is obtained for each of the non-patient patterns and the patient patterns, and the obtained probability values are displayed in colors on the square orthogonal coordinates on matrices.

The feature of the patient biosignal and the feature of the non-patient biosignal may be visualized by respectively displaying the patient patterns and non-patient patterns on the matrices, so that the difference between the non-patient biosignal and the patient biosignal may be visually identified.

Also, a mismatch pattern between the patient patterns and the non-patient patterns may be extracted by displaying the patient patterns and non-patient patterns on matrices, respectively. Also, it is easy to estimate and describe a person who has a disease depending on whether the mismatch pattern is retained.

If a disease pattern is extracted using a learning biosignal, whether a predetermined person has a disease may be estimated in a manner that inputs a predetermined person signal to the biosignal visualizing system, extracts predetermined person patterns according to the same manner as the method of extracting patterns from a biosignal, compares the extracted predetermined person patterns with the disease pattern and the non-patient patterns.

Particularly, if the predetermined person biosignal is input to the biosignal visualizing system, the predetermined person biosignal may be segmented and expressed as multiple patterns according to a predetermined period of time and condition.

After the predetermined person biosignal are segmented and expressed as multiple patterns, the value of the probability that different patterns neighboring in the multiple patterns will be adjacent to each other may be measured. After the value of the probability of adjacency between different patterns is measured, the multiple patterns may be displayed in color on a matrix according to the measured probability values.

Subsequently, the extraction unit 150 may extract whether a pattern that is identical to the disease pattern is retained in the predetermined person patterns displayed on the matrix.

That is, the patternized predetermined person biosignal is displayed on the matrix, and the matrix may be compared with the matrix on which the patient patterns are displayed, so that whether the disease pattern is retained in the predetermined person patterns may be visually identified.

Particularly, the predetermined person biosignal is different from a learning biosignal. However, if the disease pattern is included in the predetermined person patterns extracted from the predetermined person biosignal, a pattern that has the same color as that of the disease pattern may be displayed on the same location as the location in which the disease pattern is displayed, when the predetermined patterns are displayed on the matrix. As described above, since the disease pattern may be a criterion for determining whether a disease exists, it is estimated that the predetermined person has a disease based on the fact that a pattern that has the same color as that of the disease pattern is displayed on the same location as the location in which the disease pattern is displayed. That is, whether the predetermined person has a disease may be diagnosed by expressing the patternized predetermined person biosignal on the matrix and comparing the same with a learning biosignal.

Unlike the above, in order to make a diagnosis in real time in association with a predetermined person biosignal which is input in real time to a device to which the biosignal visualizing system is applied, whether the defined disease pattern appears in the patternized predetermined person biosignal may be determined without implementing the predetermined person biosignal as a matrix. That is, if the disease pattern appears in the patternized predetermined person biosignal, it is determined that the predetermined person has a disease. Accordingly, prediction and diagnosis of a disease of the predetermined person may be possible in real time.

In order to estimate whether a predetermined person has a disease by determining whether a pattern that is the same as the disease pattern is included in the predetermined person patterns of the predetermined person, the biosignal visualizing system may further include a prediction unit 160 configured to predict a disease of a predetermined person.

In order to segment a learning biosignal and a predetermined person biosignal and to express them as patterns, the learning biosignal and the predetermined person biosignal may need to be segmented based on a predetermined unit size.

To this end, the biosignal visualizing system may further include a unit determination unit 122 configured to segment a biosignal according to a predetermined unit, and a pattern assignment unit 124 configured to assign a pattern to each predetermined unit.

In this instance, in the process of patterninzing a biosignal, the number of patterns needed may increase as a predetermined unit size, which is used for segmenting the biosignal, increases.

Generally, a biosignal may be distributed to be close to the coordinate 0. Therefore, a biosignal adjacent to the coordinate 0 may be segmented in detail. Accordingly, a large number of patterns are assigned to data distributed close to the coordinate 0 (most data belongs to this case), and a large number of patterns may be obtained. A small number of patterns are assigned to data that is distributed furthest away from the coordinate 0, and a small number of patterns may be obtained.

Particularly, referring to area B of FIG. 2B and the Gaussian curve of FIG. 3, a change in the width is densely expressed as a coordinate is closer to the coordinate 0 and a change in the width is broadly expressed as a coordinate is further away from the coordinate 0. As described above, since a biosignal is generally distributed close to the coordinate 0, the purpose of the above-mentioned configuration is to patternize a large amount of data which is close to the coordinate 0, wherein most data corresponds to this case.

To this end, previous statistical analysis associated with the overall biosignal is needed. That is, by identifying the distribution of the Gaussian curve of a biosignal, a unit interval used for segmenting the biosignal may be determined. The unit interval for segmenting a biosignal which is obtained by the previous statistical analysis may be changed according to the type of biosignal, equipment (e.g., the resolution of a sensor or the like) that measures a biosignal, and the like, and an optimal variable may be determined via experimentation of an experimenter that measures a biosignal.

As described above, if a non-patient biosignal and a patient biosignal are patternized and displayed in color on square orthogonal coordinates, non-patient patterns and patient patterns may be visualized, and a disease pattern may be extracted from the visualized non-patient patterns and the patient patterns. By extracting whether the disease pattern exists, a pattern effective for predicting a disease and making a diagnosis may be visually identified as detailed information, which is advantageous.

In addition, after learning the non-patient patterns and the patient patterns, the system compares the patient patterns and the non-patient patterns with predetermined person patterns, obtained by patterninzing a predetermined person biosignal and displaying the same in color on square orthogonal coordinates, identifies whether the disease pattern is included in the predetermined person patterns, and identifies detailed information associated with whether the predetermined person has a disease.

Hereinafter, a process of predicting a disease of a predetermined person using patterns extracted from a learning biosignal and a predetermined person biosignal, will be described.

Before providing a detailed description thereof, note that a learning biosignal is classified as a patient biosignal and a non-patient biosignal when patterns are extracted according to the above-described process, and the patient biosignal and the non-patient biosignal may be expressed as patient patterns and non-patient patterns, respectively.

After the non-patient patterns and patient patterns are obtained, the value of the probability of adjacency between patterns may be obtained for each of the non-patient patterns and patient patterns. The non-patient patterns and the patient patterns may be displayed in different colors on matrix square coordinates according to the obtained probability values.

Subsequently, the biosignal visualizing system segments a predetermined person biosignal input to the system, obtains predetermined person patterns, obtains the value of the probability of adjacency between the obtained predetermined person patterns, and display the patterns in different colors according to the obtained probability values.

If the predetermined person patterns are obtained, it is determined whether a disease pattern extracted using the non-patient patterns and patient patterns is identified from the predetermined person patterns. For example, when the non-patient patterns and the patient patterns are displayed on matrices, a mismatch pattern may be extracted from the non-patient patterns and the patient patterns. The extracted mismatch pattern is referred to as a disease pattern associated with a disease that a patient has. The disease pattern may be used as a criterion for estimating whether a predetermined person is a patient depending on whether a pattern that is identical to the disease pattern is retained in the predetermined person patterns extracted from a biosignal of the predetermined person.

As described above, if a disease pattern is extracted using a learning biosignal, whether a predetermined person has a disease may be estimated in a manner that inputs a predetermined person signal to the biosignal visualizing system, extracts predetermined person patterns according to the same manner as the method of extracting patterns form a biosignal, compares the extracted predetermined person patterns with the disease patterns and the non-patient patterns.

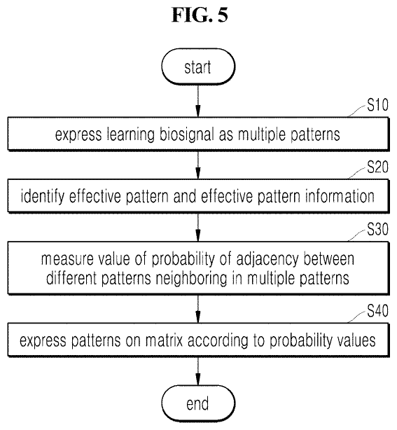

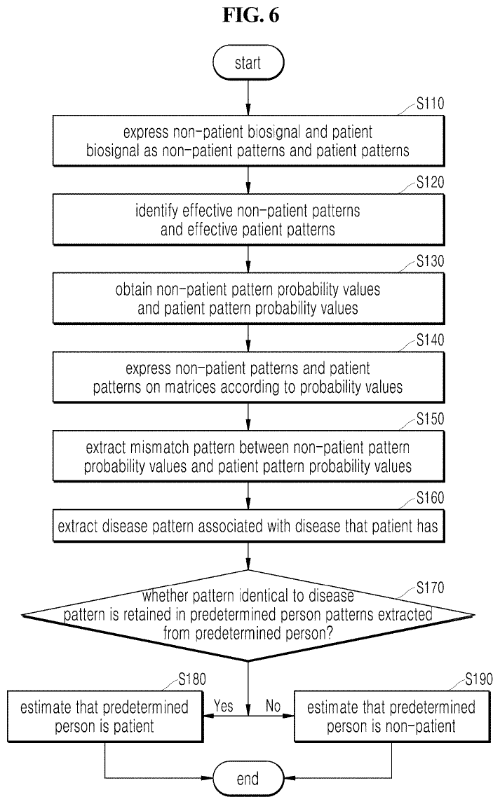

Hereinafter, a biosignal visualizing method using a biosignal visualizing system according to an embodiment of the present disclosure will be described with reference to FIGS. 5 and 6.

The biosignal visualizing method via deep learning expresses a learning biosignal, which may be learned, as multiple patterns according to a predetermined condition in operation S10.

Particularly, if a learning biosignal for learning a disease is input, the method may not learn the input learning biosignal as it is, but may simplify and learn the same. To this end, if a learning biosignal is input, the method may segment and patternize the learning biosignal according to a predetermined time and condition

The condition used for segmenting a learning biosignal may be one of the various conditions according to the type of biosignal, the type of disease to be predicted, and the like, and an embodiment of the present disclosure will describe an example of performing segmentation according to a predetermined time will be described.

In the process of patterninzing a learning biosignal, a pattern may be differently expressed according to the type of biosignal (e.g., brainwaves, ECG, and the like), a condition used for pattern segmentation, and the like.

To this end, an input learning biosignal may be analyzed, and a condition used for patterninzing the learning biosignal may be set according to the analyzed result. For example, a pattern and a variation used for determining a learning biosignal unit may be determined in order to patternize a learning biosignal.

By analyzing a learning biosignal unit according to the determined condition, a Gaussian curve (normal distribution) may be obtained. In this instance, in the obtained Gaussian curve, baseN denotes the number of patterns to be applied. It may be configured to maximize the number of patterns distributed in a part close to the center 0 of the Gaussian curve, and to minimize the number of patterns distributed in a part close to the edge of the Gaussian curve.

If the learning biosignal is patternized, whether the multiple patterns are effective patterns, and pattern information of an effective pattern is identified in operation S20.

Particularly, which pattern is an effective pattern, that is, a pattern that has information used for making a diagnosis or estimating a disease is identified among the multiple patterns.

Also, information associated with the identified effective pattern may be identified. In order to identify the information associated with the effective pattern, the information retained in the identified effective pattern may be identified via a pattern information storage unit generated during the process of patterninzing the learning biosignal (e.g., a dictionary that stores information indicated by different patterns).

For example, it is assumed that the learning biosignal is patternized as “ . . . 14C512EEEB . . . ” and <<EB>> thereof is identified as being an effective pattern. Here, it is assumed that <<EB>> is an effective pattern, which may be identified when a biosignal of a dementia patient is patternized. The information may be stored in the pattern storage unit, and the identification unit 170 compares pattern information stored in the pattern information storage unit with multiple patterns extracted from the learning biosignal, and may identify whether an effective pattern <<EB>> is included in the learning biosignal. If it is identified that the learning biosignal includes the pattern <<EB>>, information associated with the effective pattern <<EB>> may be identified based on the information stored in the pattern information storage unit.

Subsequently, the value of the probability that different patterns neighboring in the multiple patterns will be adjacent to each other may be measured in operation S30.

As described above, if it is assumed that a patternized learning biosignal is “... 14C512EEEB . . . ”, the value of the probability that pattern E and pattern E will be adjacent to each other, the value of the probability that pattern E and pattern B will be adjacent to each other, and the like may be measured.

After the values of the probability of adjacency between different patterns are measured, the measured patterns may be displayed on a matrix including columns and rows according to measured probability values in operation S40.

Particularly, the multiple patterns may be displayed in color on a matrix according to the values of the probability of adjacency between different patterns. That is, if it is assumed that the probability that pattern A and pattern A will be adjacent to each other is 1, the probability that pattern A and pattern B will be adjacent to each to other is less than the probability that pattern A and pattern A will be adjacent to each other. The probability that pattern A and pattern C will be adjacent to each other is less than the probability that pattern A and pattern B will be adjacent to each other.

To display the measured probability values on the matrix partitioned by columns and rows, the matrix is gridded to include coordinates. The coordinates may include coordinates A to Z, may include coordinates 0 to 9, or may include coordinates based on combinations of numbers and English patterns.

The matrix gridded to include coordinates may include square orthogonal coordinates, and the value of the probability of adjacency of each pattern may be displayed in color on the square orthogonal coordinates. For example, it is assumed that pattern A is a start coordinate on the square orthogonal coordinates and the value of the probability that pattern A and pattern A will be adjacent to each other is 1, the value of the probability that pattern A and pattern A will be adjacent to each other may be displayed in red on the square orthogonal coordinates. The value of the probability that pattern A and pattern B will be adjacent to each other may be different from the value of the probability that pattern A and pattern A will be adjacent to each other, and the value of the probability that pattern A and pattern B will be adjacent to each other may be displayed in blue, which is different from the color corresponding to the value of the probability that pattern A and pattern A will be adjacent to each other, on the square orthogonal coordinates.

The biosignal visualizing system according to an embodiment of the present disclosure may display multiple patterns in different colors according to a predetermined condition, when displaying the patterns on a matrix.

A learning biosignal may be classified as a non-patient biosignal and a patient biosignal. A process of obtaining a disease pattern from a learning biosignal which is classified as a non-patient biosignal and a patient biosignal, and a process of estimating whether a predetermined person biosignal retains information associated with a disease based on a disease will be described with reference to FIG. 6.

Non-patient patterns and patient patterns may be obtained by expressing the non-patient biosignal and the patient biosignal as multiple patterns according to a predetermined condition in operation S110.

After the non-patient patterns and patient patterns are obtained, whether the multiple obtained non-patient patterns and patient patterns are effective patterns may be determined, and effective non-patient pattern information and effective patient pattern information may be obtained from the non-patient patterns and the patient patterns, respectively, in operation S120.

In order to identify the information associated with the effective pattern, the information retained in the identified effective pattern may be identified via a pattern information storage unit generated during the process of patterninzing the learning biosignal (e.g., a dictionary that stores information indicated by different patterns).

That is, it is assumed that the learning biosignal is patternized as “. . . 14C512EEEB . . . ”, and pattern <<EB>> thereof is identified as being an effective pattern. Here, it is assumed that <<EB>>is an effective pattern, which may be identified when a biosignal of a dementia patient is patternized. The information may be stored in the pattern storage unit, and the identification unit 170 may compare pattern information stored in the pattern information storage unit with the multiple patterns extracted from the learning biosignal, and may identify whether the effective pattern <<EB>> is retained in the learning biosignal. If it is identified that the learning biosignal includes the pattern <<EB>>, information associated with the effective pattern <<EB>> may be identified based on the information stored in the pattern information storage unit.

After determining whether the patient patterns and the non-patient patterns are effective patterns, the method may obtain a non-patient pattern probability value indicating the probability of adjacency for each of the multiple obtained non-patient patterns and a patient pattern probability value indicating the probability of adjacency for each of the multiple obtained patient patterns in operation S130.

Particularly, if it is assumed that the patternized learning biosignal is “. . . 14C512EEEB . . . ”, the value of the probability that pattern E and pattern E will be adjacent to each other, the value of the probability that pattern E and pattern B will be adjacent to each other, and the like may be measured.

In this instance, the value of the probability may be measured in consideration of the order of neighboring patterns, according to a condition. For example, the value of the probability that pattern E and pattern B will be adjacent to each other may be measured only if the pattern E and pattern B are sequentially identified.

Unlike the above, only the value of the probability that different patterns will be adjacent to each other may be measured without taking into consideration the order of patterns. That is, only the value of the probability that pattern E and pattern B will be adjacent to each other may be measured by assuming that the case in which the patterns are aligned in the order of E and B, and the case in which the patterns are aligned in the order of B and E are considered the same.

The condition used for obtaining a probability value may be different depending on the type of biosignal, the number of patterns, and the like, and the present disclosure is not limited by the condition used for obtaining a probability value.

Subsequently, the method may respectively display the non-patient pattern probability values and the patient patterns probability values on matrix square coordinates in operation S140.

In this instance, the value of the probability that different patterns will be adjacent to each other may be measured on condition that measurement is performed according to the order of different patterns, and the value of the probability that different patterns will be adjacent to each other may be measured on condition that measurement is performed irrespective of the order of different patterns.

If the probability values measured according to the conditions are displayed on matrices, the matrices which are different in shape according to the conditions may be obtained.

For example, a matrix obtained on condition that measurement is performed without taking into consideration the order of different patterns may be implemented as a matrix which is symmetrical about a diagonal like a matrix illustrated in FIG. 4.

If an insufficient number of patterns are obtained, a probability value is measured without taking into consideration the order of patterns, so as to increase the number of patterns and to sufficiently visualize a biosignal.

Unlike the same, a matrix obtained on condition that a probability value is measured in the order of different patterns may be implemented as a matrix which is not symmetrical.

Meanwhile, the probability that pattern E and pattern B will be adjacent to each other and the probability that pattern B and pattern E will be adjacent to each other may be different from each other when a probability value is measured according to the order of patterns. If an effective pattern is <<EB>>, accurate information associated with an effective pattern (effective pattern information) may be obtained, which is advantageous.

By displaying the non-patient pattern probability values and the patient pattern probability values on matrix square coordinates, a part (an area marked by a circle in FIG. 4B, a mismatch pattern) corresponding to a mismatch between the non-patient pattern probability values and the patient pattern probability values may be extracted in operation S150.

Particularly, after obtaining a mismatch pattern between a first matrix on which non-patient patterns are displayed and a second matrix on which the patient patterns are displayed, the patient pattern probability value of the mismatch pattern may be identified and a pattern associated with the identified probability value may be extracted. That is, the name of the pattern having the probability value of the mismatch pattern may be extracted.

The mismatch pattern may be extracted by using an exclusive-or (XOR) operation. The XOR operation is an operation that gives a result of 0 when input variables have the same bit, and otherwise, it gives a result of 1. The mismatch pattern may be extracted based on an operation that gives result of 1.

As described above, a mismatch pattern may be extracted using extracted non-patient patterns and patient patterns, and the extracted mismatch pattern is referred to as a disease pattern associated with a disease that a patient has. The disease pattern may be used as a criterion for estimating whether a predetermined person is a patient depending on whether the pattern same as the disease pattern is included in predetermined person patterns extracted from a biosignal of a predetermined person in operation S170.

Accordingly, a patient biosignal and a non-patient biosignal are segmented, non-patient biosignal segments and patient biosignal segments are patternized, the value of the probability of adjacency is obtained for each of the non-patient patterns and the patient patterns, and the obtained probability values are displayed in color on the square orthogonal coordinates on matrices.

The feature of the patient biosignal and the feature of the non-patient biosignal may be visualized by respectively displaying the patient patterns and non-patient patterns on matrices, so that the difference between the non-patient biosignal and the patient biosignal are visually identified.

Also, a mismatch pattern may be extracted from the patient patterns and the non-patient patterns by respectively displaying the patient patterns and non-patient patterns on matrices. Also, it is easy to estimate and describe a person who has a disease depending on whether a mismatch pattern is retained.

As described above, if a disease pattern is extracted using a learning biosignal, whether a predetermined person has a disease may be estimated in a manner that inputs a predetermined person signal to the biosignal visualizing system, extracts predetermined person patterns according to the same manner as the method of extracting patterns form a biosignal, compares the extracted predetermined person patterns with the disease pattern and the non-patient patterns.

Particularly, if the predetermined person biosignal is input to the biosignal visualizing system, the predetermined person biosignal may be segmented and expressed as multiple patterns according to a predetermined period of time and condition.

After the predetermined person biosignal are segmented and expressed as multiple patterns, the value of the probability that different patterns neighboring in the multiple patterns will be adjacent to each other may be measured. After the values of the probability of adjacency between different patterns are measured, the multiple patterns may be displayed in color on a matrix according to the measured probability values.

Subsequently, whether a pattern that is identical to the disease pattern is retained in the predetermined person patterns displayed on the matrix may be extracted.

That is, the patternized predetermined person biosignal is displayed on the matrix, and the matrix may be compared with the matrix on which the patient patterns are displayed, so that whether the disease pattern is retained in the predetermined person patterns may be visually identified.

Particularly, the predetermined person biosignal is different from a learning biosignal. However, if the predetermined person patterns extracted from the predetermined person biosignal include the disease pattern, a pattern that has the same color as that of the disease pattern may be displayed on the same location as the location in which the disease pattern is displayed, when the predetermined person patterns are displayed on a matrix. As described above, since the disease pattern may be a criterion for determining whether a disease exists, it is estimated that the predetermined person has a disease in operation S180 and S190, based on the fact that the pattern that has the same color as that of the disease pattern is displayed on the same location as the location in which the disease pattern is displayed.

As described above, if a non-patient biosignal and a patient biosignal are patternized, and displayed in color on square orthogonal coordinates, the non-patient patterns and the patient patterns may be visualized, and a disease pattern may be extracted from the visualized non-patient patterns and patient patterns. By extracting whether the disease pattern exists, a pattern effective for predicting a disease and making a diagnosis may be visually identified as detailed information, which is advantageous.

In addition, after learning the non-patient patterns and the patient patterns, the method compares the patient patterns and the non-patient patterns with predetermined person patterns, obtained by patterninzing a predetermined person biosignal and displaying the same in color on square orthogonal coordinates, identifies whether the disease pattern is retained in the predetermined person patterns, and identifies detailed information associated with whether the predetermined person has a disease.

The implementations of the functional operations and subject matter described in the present disclosure may be realized by a digital electronic circuit, by the structure described in the present disclosure, and the equivalent including computer software, firmware, or hardware including, or by a combination of one or more thereof. Implementations of the subject matter described in the specification may be implemented in one or more computer program products, that is, one or more modules related to a computer program command encoded on a tangible program storage medium to control an operation of a processing system or the execution by the operation.

A computer-readable medium may be a machine-readable storage device, a machine-readable storage substrate, a memory device, a composition of materials influencing a machine-readable radio wave signal, or a combination of one or more thereof.

In the specification, the term “system” or “device”, for example, covers a programmable processor, a computer, or all kinds of mechanisms, devices, and machines for data processing, including a multiprocessor and a computer. The processing system may include, in addition to hardware, a code that creates an execution environment for a computer program when requested, such as a code that constitutes processor firmware, a protocol stack, a database management system, an operating system, or a combination of one or more thereof.

A computer program (also known as a program, software, software application, script, or code) can be written in any form of programming language, including compiled or interpreted languages, declarative or procedural languages, and it can be deployed in any form, including as a stand-alone program or module, a component, subroutine, or another unit suitable for use in a computer environment. A computer program may, but need not, correspond to a file in a file system. A program can be stored in a single file provided to the requested program, in multiple coordinated files (for example, files that store one or more modules, sub-programs, or portions of code), or in a portion of a file that holds other programs or data (for example, one or more scripts stored in a markup language document). A computer program can be deployed to be executed on one computer or on multiple computers that are located at one site or distributed across a plurality of sites and interconnected by a communication network.

A computer-readable medium suitable for storing a computer program command and data includes all types of non-volatile memories, media, and memory devices, for example, a semiconductor memory device such as an EPROM, an EEPROM, and a flash memory device, and a magnetic disk such as an external hard disk or an external disk, a magneto-optical disk, a CD-ROM, and a DVD-ROM disk. A processor and a memory may be added by a special purpose logic circuit or integrated into the logic circuit.

The implementations of the subject matter described in the specification may be implemented in a calculation system including a back-end component such as a data server, a middleware component such as an application server, a front-end component such as a client computer having a web browser or a graphic user interface which can interact with the implementations of the subject matter described in the specification by the user, or all combinations of one or more of the back-end, middleware, and front-end components. The components of the system can be mutually connected by any type of digital data communication such as a communication network or a medium.

While the specification contains many specific implementation details, these should not be construed as limitations to the scope of any disclosure or of what may be claimed, but rather as descriptions of features that may be specific to particular embodiments of particular disclosures. Certain features that are described in the specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

In addition, in the specification, the operations are illustrated in a specific sequence in the drawings, but it should be understood that the operations are not necessarily performed in the shown specific sequence or that all shown operations are necessarily performed in order to obtain a preferable result. In a specific case, multitasking and parallel processing may be preferable. Furthermore, it should not be understood that a separation of the various system components of the above-mentioned implementation is required in all implementations. In addition, it should be understood that the described program components and systems usually may be integrated in a single software package or may be packaged in a multi-software product.

As described above, specific terms disclosed in the specification do not intend to limit the present disclosure. Therefore, while the present disclosure was described in detail with reference to the above-mentioned examples, a person skilled in the art may modify, change, and transform some parts without departing a scope of the present disclosure. The scope of the present disclosure is defined by the appended claims to be described later, rather than the detailed description. Accordingly, it will be appreciated that all modifications or variations derived from the meaning and scope of the appended claims and their equivalents are included in the range of the present disclosure.

What is claimed is:

1. A system for visualizing a biosignal, the system comprising:
a pattern expression unit configured to express a learning biosignal as multiple patters according to a predetermined condition;
an identification unit configured to determine whether the multiple patterns are effective patterns, and to identify pattern information of the effective patterns;
a measurement unit configured to measure a value of a probability that different patterns neighboring in the multiple patterns are adjacent to each other; and
a display unit configured to display the multiple patterns on a matrix including columns and rows, according to the probability values.

2. The system of claim 1, wherein the pattern expression unit is configured to obtain non-patient patterns and patient patterns by expressing a non-patient biosignal and a patient biosignal of the learning biosignal as multiple patterns according to a predetermined condition,
wherein the identification unit is configured to determine whether the non-patient patterns and the patient patterns are effective patterns, and to identify effective non-patient pattern information and effective patient pattern information respectively from the non-patient patterns and the patient patterns,
wherein the measurement unit is configured to obtain a non-patient pattern probability value indicating a probability that different patterns neighboring in the multiple non-patient patterns are adjacent to each other, and a patient pattern probability value indicating a probability that different patterns neighboring in the multiple patient patterns are adjacent to each other, and
wherein the display unit is configured to display the non-patient pattern probability value and the patient pattern probability value on the matrix.

3. The system of claim 2, further comprising:
an extraction unit configured to extract a mismatch pattern where the non-patient pattern probability value and the patient pattern probability value do not match, so as to extract a disease pattern associated with a disease that a patient has.

4. The system of claim 3, wherein the extraction unit is configured to extract the mismatch pattern using an exclusive-or (XOR) operation

5. The system of claim 3, wherein the pattern expression unit is configured to obtain predetermined person patterns by expressing a predetermined person biosignal as multiple patterns according to a predetermined condition,
wherein the identification unit is configured to identify whether the predetermined person patterns are effective patterns, and to identify effective predetermined person pattern information from the predetermined person patterns,
wherein the measurement unit is configured to obtain a predetermined person pattern probability value indicating a probability that different patterns neighboring in the multiple predetermined person patterns are adjacent to each other,
wherein the display unit is configured to display the predetermined person pattern probability value on the matrix, and
wherein the extraction unit is configured to determine whether a pattern that matches the disease pattern is included in the predetermined person patterns.

6. The system of claim 5, further comprising:
a prediction unit configured to predict a disease of the predetermined person depending on whether the pattern that matches the disease pattern is included in the predetermined person patterns.

7. The system of claim 1, further comprising:
a pattern color determination unit configured to display the multiple patterns in different colors according to a predetermined condition

8. The system of claim 5, further comprising:
a unit determination unit configured to segment the learning biosignal and the predetermined person biosignal according to a predetermined unit size according to a predetermined condition; and
a pattern assignment unit configured to assign a pattern to each predetermined unit.

9. The system of claim 8, wherein a number of the patterns increases as the unit size used for segmenting the learning biosignal and the predetermined person biosignal increases.

10. A method of extracting an effective pattern, comprising:
expressing a learning biosignal as multiple patterns according to a predetermined condition;
identifying whether the multiple patterns are effective patterns, and identifying pattern information of the effective patterns;
measuring a value of a probability that different patterns neighboring in the multiple patterns are adjacent to each other; and
displaying the multiple patterns on a matrix including columns and rows, according to the measured probability values.

11. The method of claim 10, wherein the expressing comprises: expressing a non-patient biosignal and a patient biosignal of the learning biosignal as non-patient patterns and patient patterns,
wherein the identifying comprises: determining whether the non-patient patterns and the patient patterns are effective patterns, and identifying effective non-patient pattern information and effective patient pattern information respectively from the non-patient patterns and the patient patterns,
wherein the measuring comprises: obtaining a patient pattern probability value and a non-patient pattern probability value,
wherein the displaying comprises: displaying the non-patient pattern probability value and the patient person pattern probability value on the matrix, and
wherein the method comprises: extracting a mismatch pattern where the non-patient probability value and the patient person pattern probability value do not match, so as to extract a disease pattern associated with a disease that a patient has.

12. The method of claim 11, wherein the extracting comprises:
extracting whether a pattern that matches the disease pattern is included in predetermined person patterns extracted in association with a predetermined person

13. The method of claim 10, wherein the displaying comprises:
displaying the multiple patterns in different colors, according to a predetermined condition
